(19) United States
(12) Patent Application Publication
(10) Pub. No.: US 2013/0189236 A1
(43) Pub. Date: Jul. 25, 2013

(54) Prevention And Treatment Of Gastrointestinal Infection In Mammals

(71) Applicant: Douglas R. Ware, Chapel Hill, NC (US)

(72) Inventor: Douglas R. Ware, Chapel Hill, NC (US)

(73) Assignee: NUTRITION PHYSIOLOGY COMPANY, LLC, Guymon, OK (US)

(21) Appl. No.: 13/643,023

(22) PCT Filed: Apr. 22, 2011
(86) PCT No.: PCT/US2011/033612

§ 371 Date: Apr. 2, 2013

Related U.S. Application Data
(60) Provisional application No. 61/327,368, filed on Apr. 23, 2010.

(51) Int. Cl. A61K 35/74

(52) U.S. Cl. CPC A61K 35/747
USPC 424/93.45; 424/93.4; 424/780

(57) ABSTRACT

Methods are disclosed for the prevention and/or treatment of certain gastrointestinal (GI) diseases, such as Johne's Diseases (JD) in animals and Crohn's Disease (CD) in human. Administration of certain probiotic bacteria, such as lactic acid producing bacteria, to animals helps inhibit GI infection by Mycobacterium avium subsp. paratuberculosis (MAP). MAP is the primary pathogenic agent suspected of causing various inflammatory bowel diseases in cattle or humans.

### RELATED APPLICATIONS

This application claims priority to U.S. Patent application No. 61/327,368 filed Apr. 23, 2010, the entire content of which is hereby incorporated by reference into this application.

### BACKGROUND

1. Field of the Invention

The present disclosure pertains to use of probiotics for the prevention and/or treatment of certain gastrointestinal (GI) diseases, such as Johne's Diseases (JD) in animals and Crohn's Disease (CD) in human. More particularly, the disclosure relates to inhibition of GI infection caused by Mycobacterium avium subsp. paratuberculosis (MAP).

2. Description of the Related Art

One of the most common inflammatory bowel diseases (IBD) in cattle, Johne's Disease (JD), shares many clinical manifestations that are similar to those in the human inflammatory bowel disease Crohn's disease (CD). Johne's disease is a contagious, chronic and usually fatal infection that affects primarily the small intestine of ruminants. Johne's disease is caused by Mycobacterium avium subspecies paratuberculosis (MAP). Because a high percentage of Crohn's patients harbor MAP, it is believed that MAP is at least one of the causes of the center of Crohn's disease.

Symptoms of Johne's disease include, for example, weight loss and diarrhea with a normal appetite. Several weeks after the onset of diarrhea, a soft swelling may occur under the jaw (bottle jaw). Bottle jaw or intermandibular edema is due to protein loss from the bloodstream into the digestive tract. Animals at this stage of the disease usually do not live very long, typically a few weeks at most.

Animals are most susceptible to MAP infection during the first year of life, and signs of the infection may not become evident until years after the initial infection. Newborns most often become infected by swallowing small amounts of infected manure from the birthing environment or from the udder of the mother. In addition, newborns may become infected while in the uterus or by swallowing bacteria passed in milk and colostrum.

Although antibiotic therapy, modified diets and probiotics have been proposed or used to treat IBD, effective treatment for JD and CD has not been found. Probiotics, such as lactic acid producing bacteria have been shown to modify the immune response in mice. Chuang, et al., J. Agric. Food Chem., 2007, 55 (26), page 11080-86 (2007). However, there appear to be conflicting data as to how the immune response is modified. While Chuang et al. showed that certain Lactobacillus strains stimulate cell /proliferation and the production of interleukin(IL)-10, IL-12 and interferon (IFN)-gamma in splenocytes, Llopis et al. showed that live Lactobacillus casei significantly decreased secretion of TNF-alpha, IFN-gamma, IL-2, IL-6, IL-8, and CXCL1 by CD mucosa. Compare Chuang et al. with Abstract of Llopis et al., Inflamm Bowel Dis., Volume 15, Number 2, 275-83 (2009). It is not clear if any Lactobacillus species will be truly effective in preventing or treating MAP infections.

Probiotics other than lactic acid bacteria have been used to curtail the progression of JD. For instance, Click et al. has shown that a unique bacterium, Dietzia ssp. C79793-74, was therapeutic for adult paratuberculosis animals, and resulted in a cure rate of 37.5%. However, because bacteria of the Dietzia genus may be harmful to the animals, their use as a acceptable probiotics is limited.

### SUMMARY

The present instrumentalities advance the art by providing methods for preventing and/or treating a gastrointestinal disease caused by Mycobacterium avium subsp. paratuberculosis (MAP) infection. The gastrointestinal diseases may include but are not limited to Johne's Disease in animals, Crohn's Disease in human, or other inflammatory bowel diseases (IBD). In one embodiment, lactic acid producing bacteria or derivative thereof may be provided to animals that have been infected by MAP to help inhibit the spread of the infection. In another embodiment, lactic acid producing bacteria may be provided to animals that have not been infected by MAP to help prevent the infection.

The present disclosure provides methods for preventing or treating various gastrointestinal diseases in a subject by administering to an effective amount of at least one probiotic bacterium to the subject. Preferably, the subject has contracted the gastrointestinal disease or is living in an environment having Mycobacterium avium subsp. paratuberculosis (MAP) in its vicinity. The subject may be an animal or a human. Animals may include but are not limited to ruminants and other mammals, such as sheep, goats and cattle. Because animals or humans typically become infected by MAP through ingestion of the bacteria, a animal or a human sharing a living environment with an infected individual may have an increased chance of ingesting a MAP from the infected individual. For purpose of this disclosure, where there is a substantial chance that an animal or a human may get in contact with the body, body fluid, excretion or feces of a MAP-infected animal, such animal or human can be said to have increased susceptibility to MAP infection.

The methods of the present disclosure may optionally include a diagnostic step wherein an animal is tested to determine whether it has been infected by MAP or whether it is more susceptible to MAP infection than other animals in the same herd before administration of the probiotic bacterium to the animal. In another aspect, a mammal may be deemed to have an increased susceptibility to infection by MAP if the mammal is more susceptible to MAP infection as compared to the average susceptibility of mammals belonging to the same species. In another aspect, the diagnostic step may include testing of a herd (i.e., more than one animal) to determine if any animal in the herd has contracted the MAP. Thus, in one embodiment, animals that are in need of a treatment to prevent or to cure MAP infection are identified before probiotic bacteria are provided to such an animal. Alternatively, the lactic acid producing bacteria of the present disclosure may be supplemented to an animal without first confirming whether the animal is in need of such supplementation for the prevention or treatment of a gastrointestinal disease.

A number of different probiotics may be used for the purpose of the instant disclosure. In one embodiment, the probiotic bacterium to be fed to the mammal may be a lactic acid producing bacterium, such as, bacteria belonging to the genus of Lactobacillus or Pediococcus. Examples of the strains of the lactic acid producing bacteria include but are not limited to C28, M35, LA45, NP51 (also known as LA 51), L411, D3 or combination thereof In another embodiment, the at least one probiotic bacterium may contain a lactic acid producing bacterium and a lactic acid utilizing bacterium. For example, the at least one probiotic bacterium may contain at least one species belonging to the genus of Lactobacillus and at least one species belonging to the genus of Propionibacterium.

Lactobacillus Strains C28, M35, LA45, and LA51 strains were deposited with the American Type Culture Collection (ATCC, Manassas, Va. 20110-2209) on May 25, 2005 and have the Deposit numbers of PTA-6748, PTA-6751, PTA-6749, and PTA-6750, respectively. Lactobacillus strain L411 was deposited with the ATCC on June 30, 2005 and has the Deposit number of PTA-6820. Pediococcus acidilactici strain D3 was deposited with the ATCC on Mar. 8, 2006 and has the Deposit number of PTA-7426.

Examples of Propionibacterium freudenreichii strains may include but are not limited to the P9, PF24, P42, P93 and P99 strains. In another embodiment, the Propionibacterium freudenreichii strain is PF24. Propionibacterium strain PF24 was deposited with the ATCC on May 25, 2005 and has the Deposit numbers of PTA-6752. P9 and P42 were deposited with the ATCC on Jun. 30, 2005 and have the Deposit numbers of PTA-6821 and PTA-6822, respectively.

These deposits were made in compliance with the Budapest Treaty requirements that the duration of the deposit should be for thirty (30) years from the date of deposit or for five (5) years after the last request for the deposit at the depository, or for the enforceable life of a patent that results from this application in respective PCT member countries, whichever is longer. The strains will be replenished should it become non-viable at the Depository.

The lactic acid producing bacteria or derivatives thereof may include but are not limited to live lactic acid producing bacteria, lactic acid producing bacteria that have been inactivated (e.g., by heat or by other methods), or extract of a lactic acid producing bacterium. For purpose of this disclosure, when inactivated bacteria or extract of bacteria are used, the dosage as defined by Colony Forming Unit (CFU) refers to the CFU of the live bacteria that were used to prepare the inactivated bacteria or extract thereof.

As shown here, the probiotic bacteria of the present disclosure may modify the immune response of the subject mammal which explains, at least in part, the reduced infectivity and virulence of the MAP in animals fed with the probiotic bacteria, such as NP51. In one aspect, the amount of the probiotic bacteria to be fed to the subject is an amount effective in reducing the infectibility of the subject by the Mycobacterium avium subsp. paratuberculosis (MAP). In another aspect, the number of CD8 positive cytotoxic T cells significantly increases in animals fed with the probiotics. Preferably, sufficient number of the probiotic bacteria are administered to an animal (subject) in an amount effective to increase the frequency of cytotoxic T cells in the spleen of said subject by at least 5%, or more preferably at least 10% about 45 days after treatment as compared to the frequency of cytotoxic T cells in the spleen of untreated animal of the same breed.

In another aspect, the probiotic bacteria of the present disclosure may also reduce the MAP burden in the subject in the event of MAP infection. In a preferred embodiment, amount of the probiotic bacteria to be administered to the subject is an amount effective in reducing the MAP burden by at least 40%, 50%, or even more preferably at least 60% in at least one organ of the subject, which is preferably the liver, spleen or MLN of the subject. More preferably, the MAP burden in the liver of said animal is reduced at least 80% after a period of between 100 days to 180 days of treatment by the probiotic bacteria as compared to the MAP burden in the liver of untreated mammal of the same breed.

As shown in the Examples, the probiotic bacteria may also be capable of modifying the profile of cytokines and/or chemokines in the host subject. Preferably, the amount of the probiotic bacteria to be administered to the subject is an amount effective in increasing the levels of at least one pro-inflammatory cytokine in said mammal by at least 40%, 50%, or even more preferably 60% after a period of between 100 days to 180 days post administration of the probiotics, as compared to the levels of said at least one pro-inflammatory cytokine in untreated mammal of the same breed. The at least one pro-inflammatory cytokine is preferably selected from the group consisting of IL-12, IFN-gamma, TNF-alpha or combination thereof.

The lactic acid producing bacteria are preferably fed through oral administration together with food or drink. The dosage of the lactic acid producing bacteria is preferably between 10⁵ and 10⁸ CFU per mammal per day, and more preferably, about 10⁶ CFU per mammal per day.

In another aspect, because animals may become infected very early in life, it is preferred that young animals be supplemented with the probiotic bacteria right after birth, if practicable. In another aspect, calves may be fed the probiotic bacteria not later than 2 weeks, 4 weeks, or 6 weeks after birth.

Higher dosage of lactic acid producing bacteria has been known to reduce the number of pathogens, such as E. coli O157:H7 or Salmonella in cattle. Reduction of pathogenic bacteria in cattle, may, in turn, enhance the food safety of meat or milk generated from such cattle. In a preferred embodiment, the dosage of the lactic acid producing bacteria may be increased to between 5×10⁸ and 5×10⁹ CFU per mammal per day at the very late stage of life of the animal, such as, for example, about 40 days prior to the time when said animal is to be slaughtered. In another embodiment, the at least one probiotic bacterium suitable for the disclosed method may include the Lactobacillus strain NP51 and the Propionibacterium strain PF24.

### BRIEF DESCRIPTION OF THE DRAWINGS

FIG. 1 shows the bodyweight of the mice from Day 1 to Day 180 as measured at a 15-day interval.

FIG. 2 shows the MAP burden in the liver of mice that have been treated with VM (viable MAP), VM+HNP (heat-killed NP51) or VM+VNP (viable NP51) on Day 135.

FIG. 3 shows the MAP burden in the MLN of mice that have been treated with VM (viable MAP), VM+HNP (heat-killed NP51) or VM+VNP (viable NP51) on Day 135

FIG. 4 shows the MAP burden in the spleen of mice that have been treated with VM (viable MAP), VM+HNP (heat-killed NP51) or VM+VNP (viable NP51) on Day 135

FIG. 5 shows the average scores of acid-fast bacilli at Day 180. VM (viable MAP), HM (heat-killed MAP).

FIG. 6 shows the numbers of CD8⁺ cytotoxic T cells in different treatment groups at Day 90.

FIG. 7 shows the numbers of CD8⁺ cytotoxic T cells in different treatment groups at Day 135.

FIG. 8 shows the numbers of CD8⁺ cytotoxic T cells in different treatment groups at Day 180.

FIG. 9 shows the levels of IL-12 over the course of the MAP infection in animals fed with NP51 as compared to those in animals not fed with NP51.

FIG. 10 shows the levels of IFN-gamma over the course of the MAP infection in animals fed with NP51 as compared to those in animals not fed with NP51.

FIG. 11 shows the levels of MIG over the course of the MAP infection in animals fed with NP51 as compared to those in animals not fed with NP51.

FIG. 12 shows the levels of Keratinocyte chemoattractant response over the course of the MAP infection in animals fed with NP51 as compared to those in animals not fed with NP51.

FIG. 13 shows the histology of gastrointestinal tissues (stomach tissue) from untreated mice (upper panels) and mice treated with maltodextrin (lower panels).

FIG. 14 shows the histology of gastrointestinal tissues (stomach tissue) from mice treated with heat-killed NP51 (upper panels) and mice treated live NP51 (lower panels).

### DETAILED DESCRIPTION

There will now be shown and described methods for protecting an animal from MAP infection and for treating/curing such infection by MAP through administration of certain probiotic bacteria to the animal.

The term “infectibility” refers to the likelihood that a subject (an animal or a human) will become infected. The term “infectivity” refers the capability a microorganism possesses in infecting a subject. The term “infect” means a microorganism gain entry into a target subject and establish a significant colony size.

The terms “animal” is used in its broadest term and may include human. The term “probiotics” and “probiotic bacteria” may be used interchangeably throughout this disclosure. For purpose of this disclosure, probiotic bacteria may include lactic acid producing bacteria, among others.

In one embodiment, the lactic acid producing bacterium may be selected from the group consisting of: Bacillus subtilis, Bifidobacterium adolescentis, Bifidobacterium animalis, Bifidobacterium bifidum, Bifidobacterium infantis, Bifidobacterium longum, Bifidobacterium thermophilum, Lactobacillus acidophilus, Lactobacillus agilis, Lactobacillus alactosus, Lactobacillus alimentarius, Lactobacillus amylophilus, Lactobacillus amylovorans, Lactobacillus amylovorus, Lactobacillus animalis, Lactobacillus batatas, Lactobacillus bavaricus, Lactobacillus bifermentans, Lactobacillus bifidus, Lactobacillus brevis, Lactobacillus buchnerii, Lactobacillus bulgaricus, Lactobacillus catenaforme, Lactobacillus casei, Lactobacillus cellobiosus, Lactobacillus collinoides, Lactobacillus confusus, Lactobacillus coprophilus, Lactobacillus coryniformis, Lactobacillus corynoides, Lactobacillus crispatus, Lactobacillus curvatus, Lactobacillus delbrueckii, Lactobacillus desidiosus, Lactobacillus divergens, Lactobacillus enterii, Lactobacillus farciminis, Lactobacillus fermentum, Lactobacillus frigidus, Lactobacillus fructivorans, Lactobacillus fiuctosus, Lactobacillus gasseri, Lactobacillus halotolerans, Lactobacillus helveticus, Lactobacillus heterohiochii, Lactobacillus hilgardii, Lactobacillus hordniae, Lactobacillus inulinus, Lactobacillus jensenii, Lactobacillus jugurti, Lactobacillus kandleri, Lactobacillus kefir, Lactobacillus lactis, Lactobacillus leichmannii, Lactobacillus lindneri, Lactobacillus malefermentans, Lactobacillus mali, Lactobacillus maltaromicus, Lactobacillus minor, Lactobacillus minutus, Lactobacillus mobilis, Lactobacillus murinus, Lactobacillus pentosus, Lactobacillus plantarum, Lactobacillus pseudoplantarum, Lactobacillus reuteri, Lactobacillus rhamnosus, Lactobacillus rogosae, Lactobacillus tolerans, Lactobacillus torquens, Lactobacillus ruminis, Lactobacillus sake, Lactobacillus salivarius, Lactobacillus sanfrancisco, Lactobacillus sharpeae, Lactobacillus trichodes, Lactobacillus vaccinostercus, Lactobacillus viridescens, Lactobacillus vitulinus, Lactobacillus xylosus, Lactobacillus yamanashiensis, Lactobacillus zeae, Pediococcus acidilactici, Pediococcus pentosaceus, Streptococcus cremoris, Streptococcus diacetylactis, Streptococcus (Enterococcus) faecium, Streptococcus intermedius, Streptococcus lactis, Streptococcus thermophilus, and combinations thereof.

It is to be noted that, as used in this specification and the claims, the singular forms “a,” “an,” and “the” include plural referents unless the context clearly dictates otherwise. Thus, for example, reference to “a pathogen” includes reference to a mixture of two or more pathogens, reference to “a lactic acid producing bacterium” includes reference to bacterial cells that are lactic acid producing bacteria.

The terms “between” and “at least” as used herein are inclusive. For example, a range of “between 5 and 10” means any amount equal to or greater than 5 but equal to or smaller than 10.

The various probiotics disclosed herein are either publicly available or have been deposited with the American Type Culture Collection, Manassas, V.a 20110-2209. This deposit will be made in compliance with the Budapest Treaty requirements that the duration of the deposit should be for thirty (30) years from the date of deposit or for five (5) years after the last request for the deposit at the depository or for the enforceable life of a U.S. Patent that matures from this application, whichever is longer. All deposited materials will be replenished should it become non-viable at the depository.

The following examples illustrate the present invention. These examples are provided for purposes of illustration only and are not intended to be limiting. The strains, chemicals and other ingredients are presented as typical components or reactants, and various modifications may be derived in view of the foregoing disclosure within the scope of the invention.

### EXAMPLE 1

Prevention of Mycobacterium avium subsp. paratuberculosis (MAP) infection in mice by oral administration of a Lactobacillus acidophilus NP51

Three hundred and seventy (370) Balb/c mice, 185 males and 185 females, were kept in a pathogen-free environment in standard mouse cages with raised-wire floor. Starting at the age of 6 weeks old, these mice were fed 3-5 grams per day of sterile chow meal containing different forms of the probiotics NP51 at a dosage of about 1×10⁶ CFU per mouse per day until the end of the study. The NP51 strain was provided by Nutrition Physiology Company, LLC.

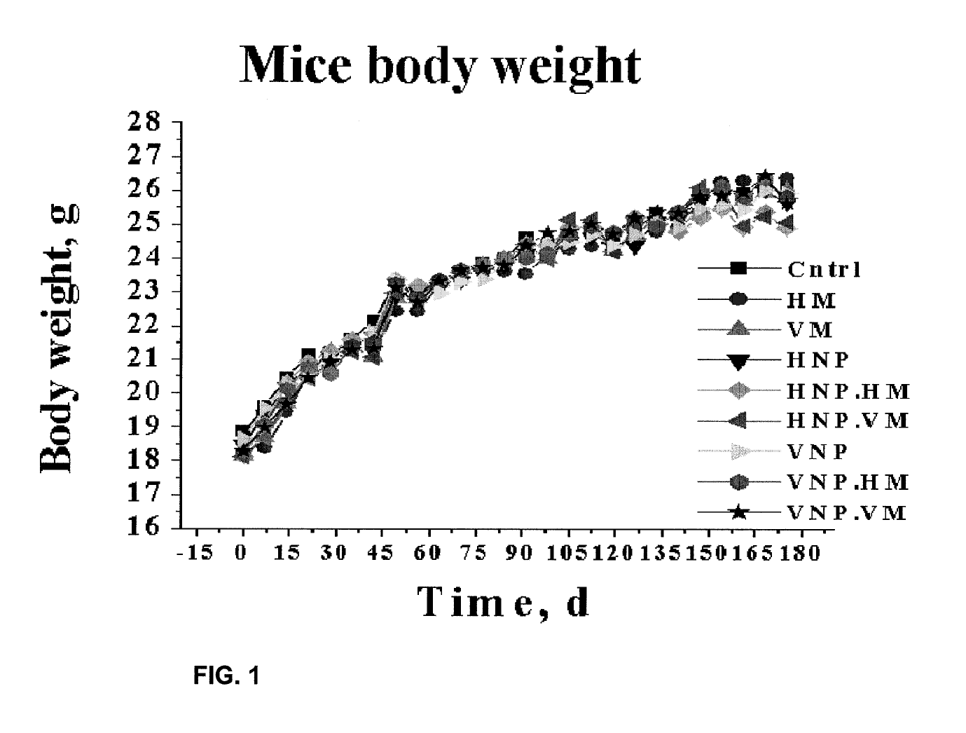

The mice were placed on a diet containing the NP51 probiotics for 45 days (Day 1 to Day 45). On Day 45, the mice were challenged with 1×10⁸ CFU of heat-killed or viable MAP through intraperitoneal injection. The mice were randomly assigned to ten treatment groups in a factorial design which include, for example, mice fed with either heat-killed or viable NP51 and mice challenged with either heat-killed or viable MAP. Ten mice from each group were euthanized at the following four time points (sampling points): Day 45, 90, 135, and 180, respectively, after the mice had been placed on a diet containing the NP51 probiotics. A summary of the 10 treatment groups and the time points is shown in Table 1. Bodyweight of the mice were monitored every 15 days from Day 1 to Day 180, and the results are shown in FIG. 1.

TABLE 1
TREATMENT GROUPS AND TIME POINTS

| | Treatment Groups | | | | | | | | | |
|---|---|---|---|---|---|---|---|---|---|---|
| Time of | Control | | | | | | Treatment | | | |
| tissue | Senti- | Cont'l + | Viable | | Heat killed | | V-NP51 + | V-NP51 + | HK-NP51 + | HK-NP51 + |
| harvest | nels | MDX | NP51 | MAP | NP51 | MAP | V-MAP | HK-MAP | V-MAP | HK-MAP |

| | | | | | | |
|---|---|---|---|---|---|---|
| Day 1 | MDX | NP51 | MDX | NP51 | MDX | Start feeding the NP51 |

| | | | | | |
|---|---|---|---|---|---|
| Day 45 | PBS | PBS | MAP | PBS | Injected MAP or PBS I.P. and euthanized |
| | | | | | 10 mice from each treatment group |

| | | |
|---|---|---|
| Day 90 | | euthanized 10 mice from each treatment group on days 90, 135, and 180 |
| Day 135 | | |
| Day 180 | 10 mice | |

MDX: Maldextrin
V: viable
HK: heat-killed

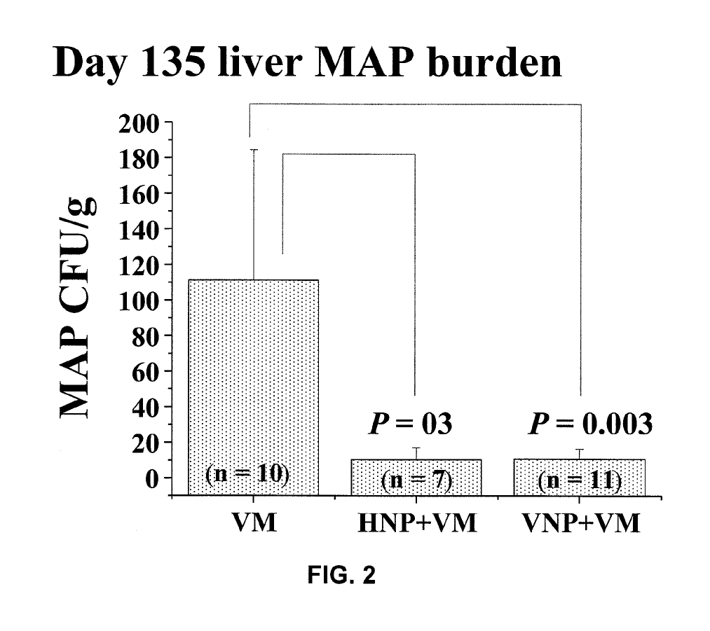

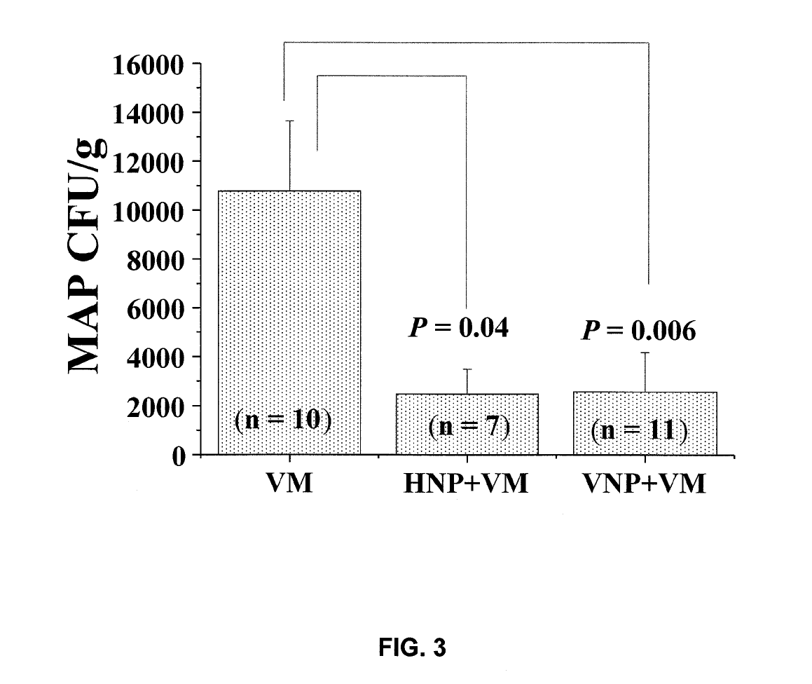

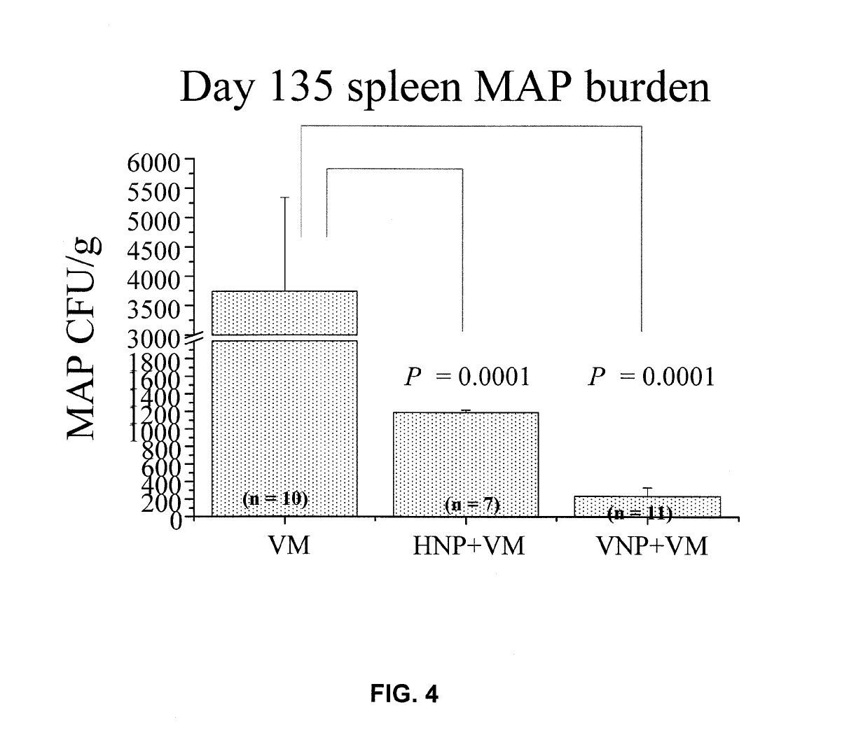

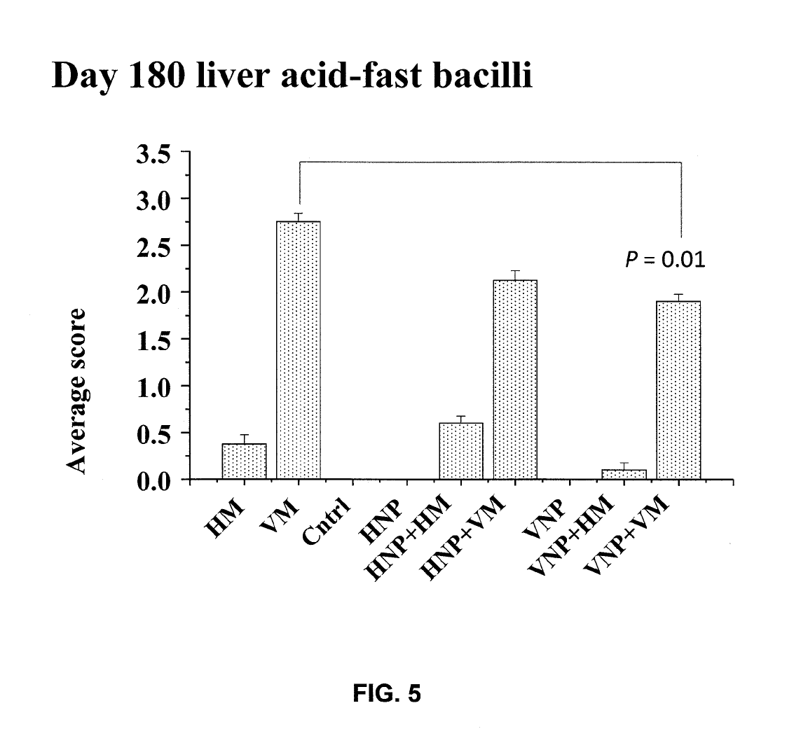

At each sampling point, tissues were excised from the mice and cultured for MAP. MAP was enumerated in livers, spleens and the peripherals, such as mesenteric lymph nodes (MLN) of the animals. The results of the MAP burden in the livers, MLN and spleens 135 days after being fed the NP51 are shown in FIG. 2, FIG. 3 and FIG. 4, respectively. H&E-stained slides of the liver tissues were examined for granulomatous reaction. In addition, Ziehl-Neelsen-stained slides from liver tissues were examined to determine whether acid-fast bacilli were present in those tissues. The average scores of acid-fast bacilli at Day 180 are shown in FIG. 5.

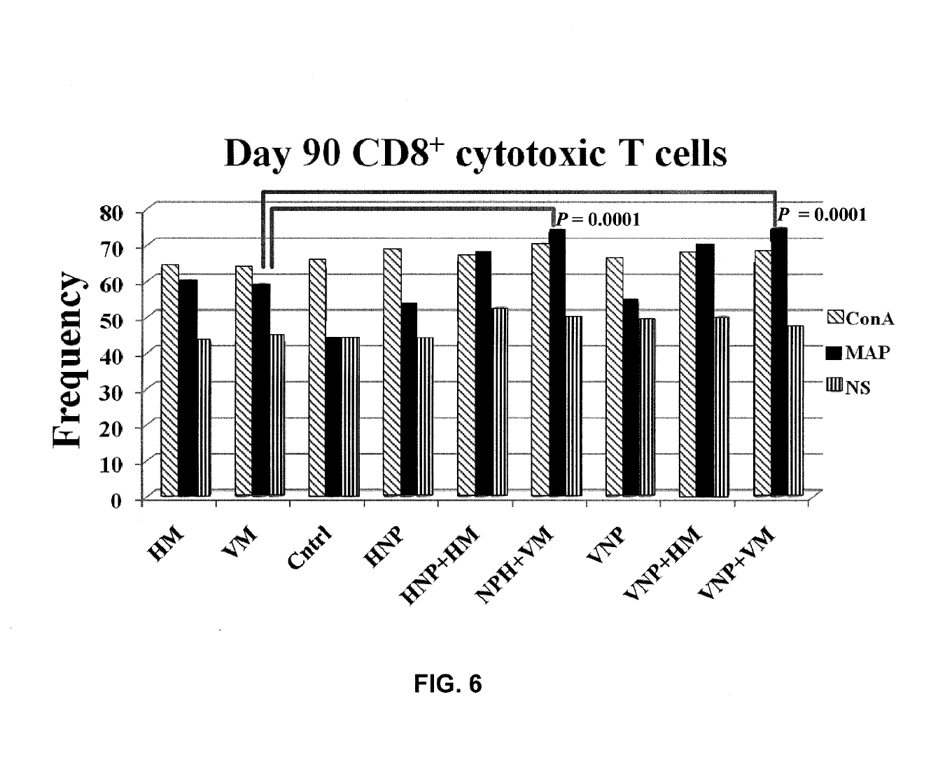

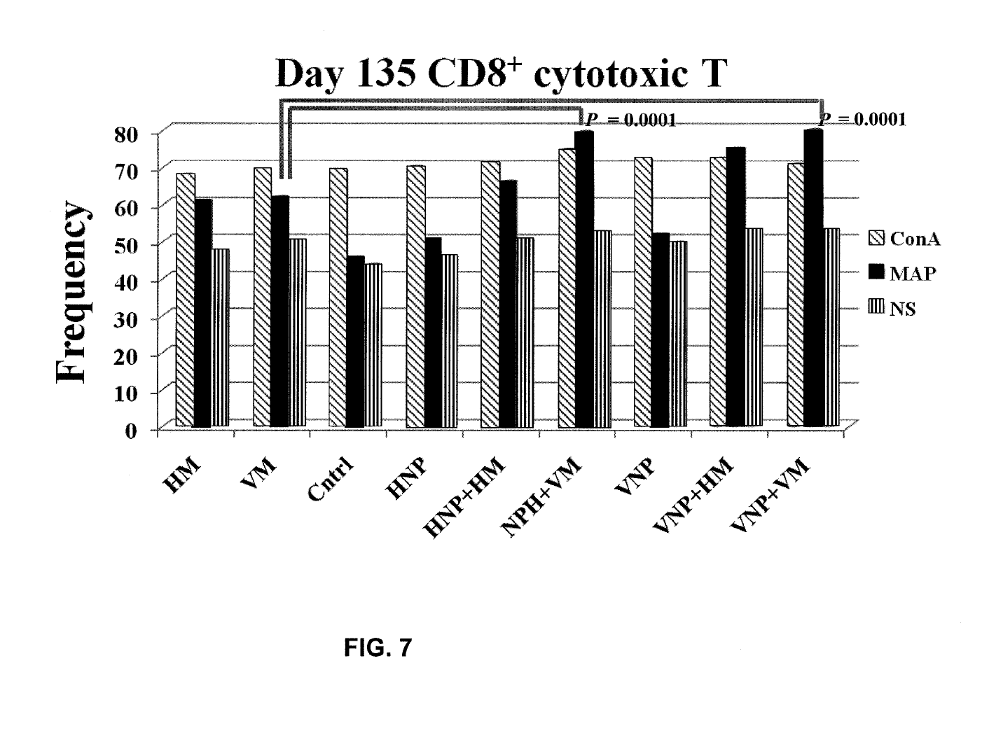

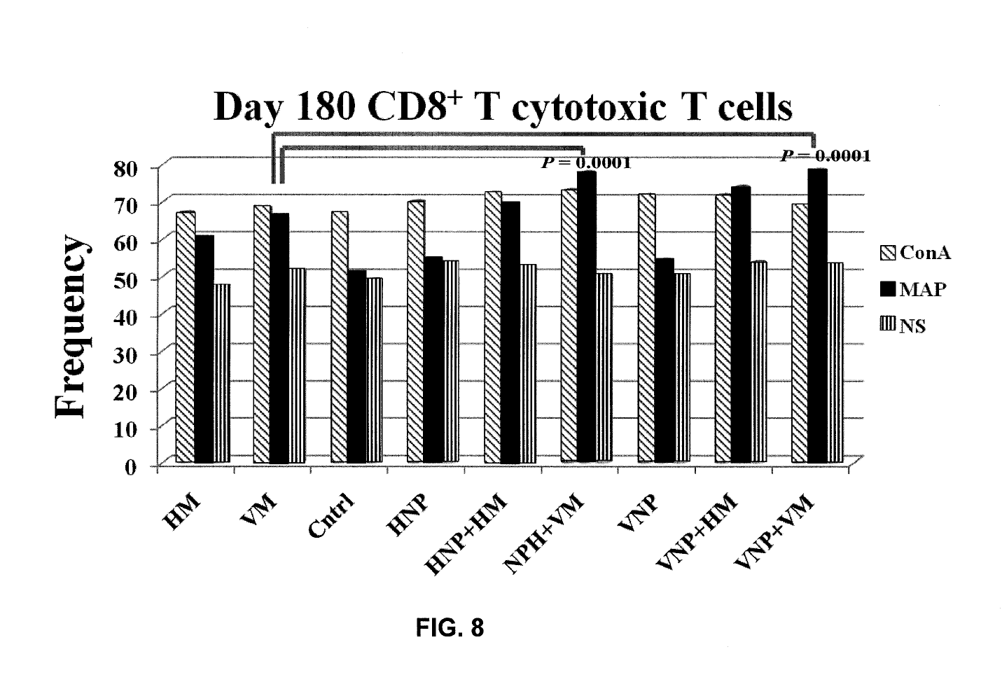

Spleens were dissected from the animals on day 45, 90, 135 and 180 and used for in vitro stimulation. More specifically, splenocytes were cultured in vitro with either MAP antigen or concanavalin A and examined for proliferation of T cells subpopulations. CD25⁺, CD4⁺, CD4⁺ CD25⁺, CD8⁺ and CD8⁺ CD25⁺ T cells were enumerated by flow cytometry. The numbers of CD8⁺ cytotoxic T cells in different treatment groups at Day 90, 135 and 180 are shown in FIG. 6, FIG. 7 and FIG. 8, respectively.

ELISA was used to quantify the following cytokines and immunoglobulins: Interleukin 12 (IL-12), IFN-gamma, IgA, IgG₁, and IgG₂ₐ. In addition, cell regulatory factors that are associated with chemotaxis recruitment of granulocytes and monocytes/macrophage were evaluated because these cell types were known to be associated with immune response for infections by intracellular pathogens such as MAP.

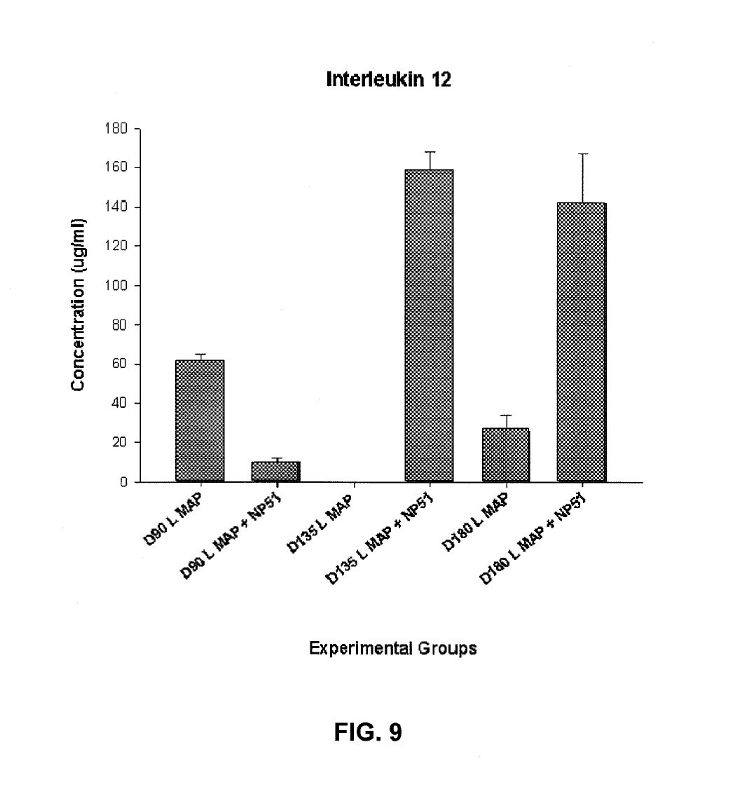

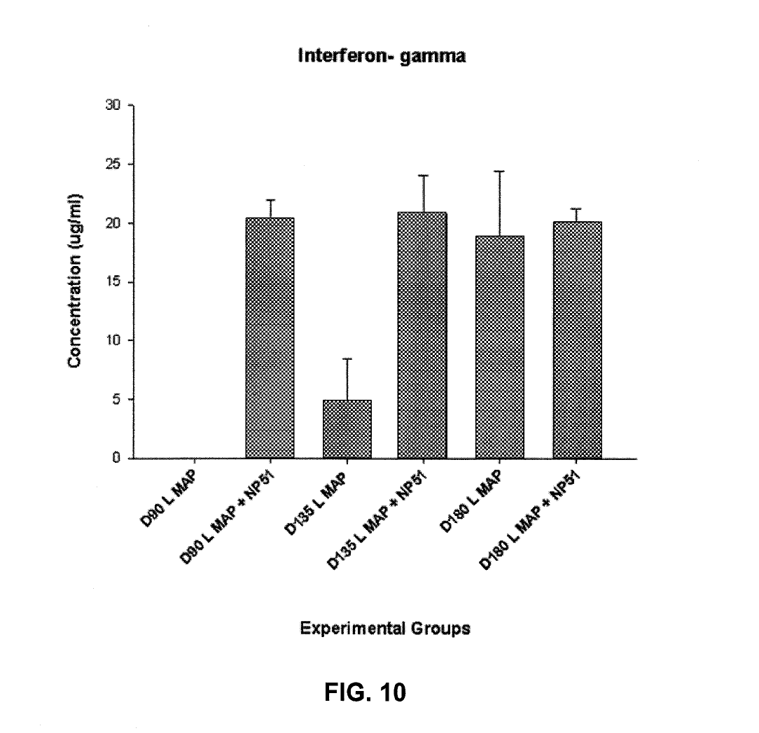

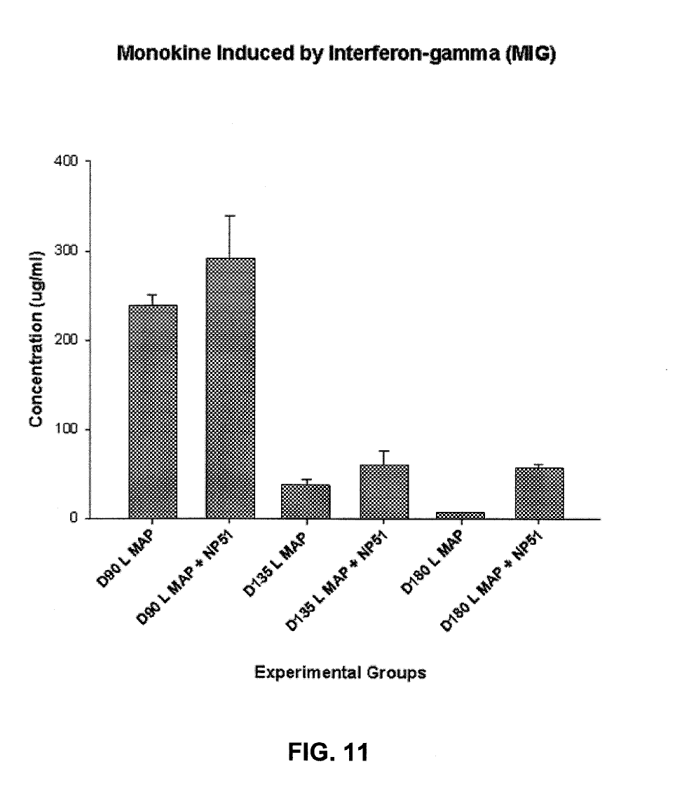

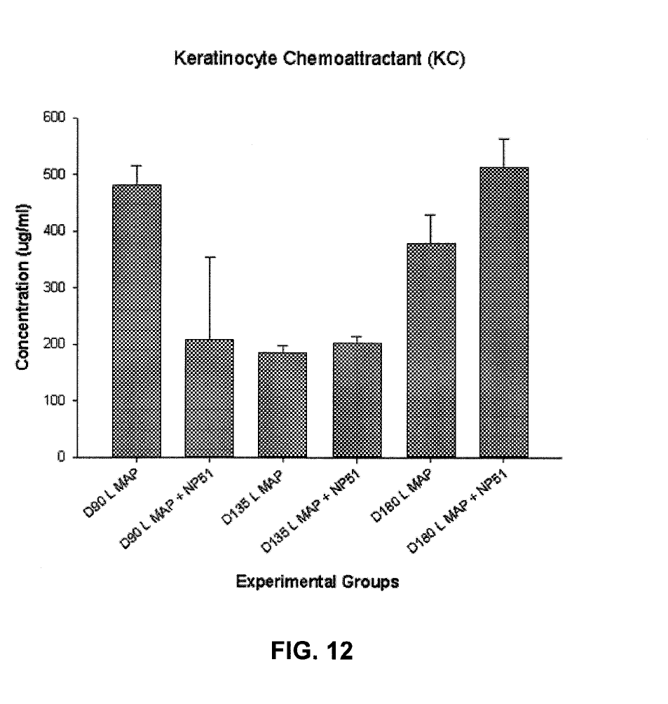

As shown in FIG. 9, IL-12 responses remained elevated over the course of the MAP infection in animals fed with NP-51 (FIG. 9). By contrast, in animals infected with MAP without NP51 supplementation, IL-12 increased during the initial MAP infection and subside overtime. The levels of IFN-gamma also increased in the presence of NP-51 in MAP infected animals, as compared to MAP infected animals without NP51 (FIG. 10). Monokine induced by IFN-gamma (MIG) also increased over time in MAP infected animals in the presence of NP51 as compared to animals with only MAP infection but no NP51 supplementation (FIG. 11). Keratinocyte chemoattractant (KC), a chemotactic factor implicated for the recruitment of neutrophils and monocytes, also increased in MAP infected mice fed with NP51, relative to MAP infected animals without NP51 (FIG. 12). These data further suggest that in the event of MAP infection, the presence of NP51, may increase immune response and migration and infiltration of cells to fend off the MAP infections. Macrophage and neutrophils are among the most notable immune cells which contribute towards preventing the spread of MAP.

Overall, feeding NP51 to mice (either heat-killed or viable) significantly increased the frequency of CD8⁺ cytotoxic T cells in spleens of mice infected with viable MAP. The levels of pro-inflammatory cytokines are also increased in animals administered the NP51 as compared to the controls. Moreover, MAP burden was decreased in the mesenteric lymph nodes, livers, and spleens of mice fed with the viable or heat-killed NP51 compared with the MAP-infected controls not fed with NP51. These results suggest that feeding NP51 may help modify the immune responses and therefore help prevent progression of MAP infection in Balb/c in mice.

Although Balb/c mice do not develop the classical symptoms of Johne's Disease in cattle, the decrease of the infectivity and the virulence of MAP observed in mice may help prevent and/or treat Johne's Disease in cattle. For instance, heat-killed or viable lactic acid producing bacteria, such as NP51, may be administered to cattle at different dosage at different stages of development. In one aspect, the lactic acid producing bacteria may be provided to the cattle prior to the time when the cattle are exposed to infectious agents that may cause Johne's Disease, such as MAP. Inhibition of the progression of MAP infection is likely to result in decrease incidence of JD in the animals. The dosage of the lactic acid producing bacteria may be adjusted according to the different bodyweight and the difference between mice and bovine animals with respect to the anatomy and physiology of their GI systems.

### EXAMPLE 2

NP51 Dosage Study in Balb/c Mice

In order to evaluate the health effects of long term supplementation of probiotics to animals, variable concentrations of a probiotic were fed to six-week old BALB/c mice over forty-five days. The influence of the probiotics on the microbial population of the gut and the histopathology of the GI tissue were compared to negative controls (no probiotics fed). The health of these mice were evaluated through histopathology analysis, which include the following tissues: gastrointestinal tissues (esophagus, small and large intestine, and stomach), liver, and spleen. Bacterial floral concentrations in fecal pellets, and gut tissues were also analyzed for the effects on microbial population diversity.

Over the course of forty-five days, 80 mice (10 mice per treatment group) were fed fresh sterilized mouse chow with either no probiotics, an inert filler maldextrin, a live probiotic (NP51) at concentrations of 1×10⁴, 10⁵, or 10⁶ CFU/g chow, or identical concentrations of the same probiotic NP51 that had been heat-killed. Sterilized mouse chow was mixed with fresh probiotic on a daily basis for feedings.

Weight and fecal pellets from each treatment group were taken daily. For this study, fecal pellets samples were ground with mortar and pestle in liquid nitrogen and kept at -80° C. for preservation of DNA samples for analysis with real-time PCR. Real-time PCR analysis were conducted to monitor the concentrations of the following flora: Enterococcus fecalis or E. faceium and Staphylococcus aureaus. Three random sample sets, for the experimental conditions from each week, were evaluated on Brain Heart Infusion (BHI) Agar and DE MAN, ROGOSA and SHARPE (MRS) agar and the total CFU/ml was recorded.

No significant difference (P<0.05) in weight gain was observed during the study period between mice in the control versus treatment groups. The total bacterial concentration observed between control conditions and treatment groups did not significantly P<0.05 change over the course of the study. However, mice fed the live probiotic, at all 3 concentrations (1×10⁴, 10⁵, or 10⁶ CFU/g chow), showed a significant decrease in the presence of Enterococcus fecalis. These results indicate the effects of acute probiotic consumption to the host and their natural gut flora. In addition, these results show that probiotic consumption can change the population of the host's natural flora over time even though the total population size may not change significantly.

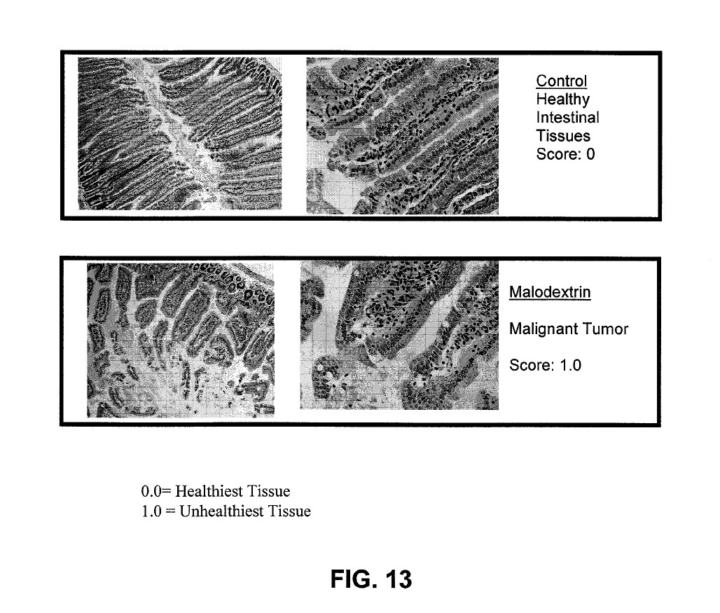

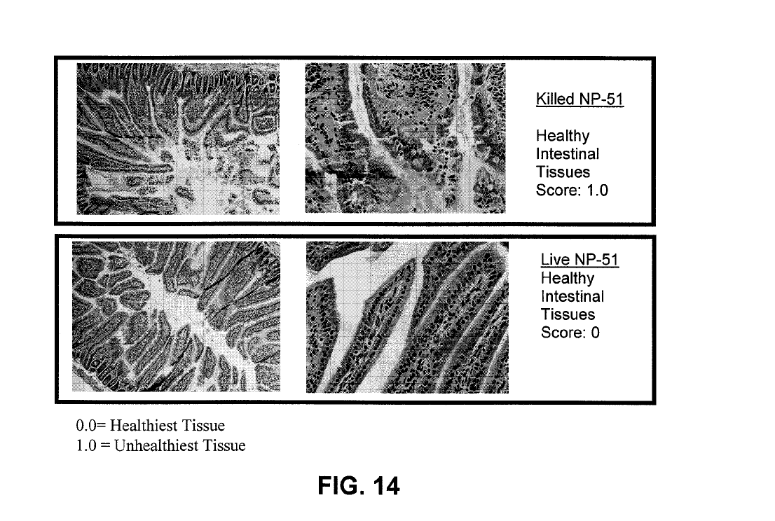

Gastrointestinal Tissue (stomach tissue) from the mice were stained using the Hemotoxylin & Eosin Stain (H& E Stain), and the results are shown in FIGS. 13 and 14. The histopathology of stomach tissues shows that animals fed NP-51 have tissue scores similar to those fed feed with no additives. These results suggest that NP-51 does not produce harmful event in the gastrointestinal tissues of mice fed NP-51 daily at the dosage used.

Cytokine and gastrointestinal gene expression were evaluated through real-time PCR analysis of RNA expression relative to control. Real-time PCR analysis was also used for fecal pellet and guts content analysis for changes in flora, host tissue, and MAP infection. ELISA analysis of IL-10, IL-12b, IL-1, TNF-α, TGF-β, and NF-κB were evaluated to distinguish immune response from early to chronic disease. Cell adhesion molecule expression (ICAM) from small intestinal tissue was also evaluated through immunohistochemistry to determine variation in receptor expression between treatment groups. Table 2 list some of the results showing changes in cytokine and other gene expression. (⇑) indicates up-regulation of gene expression relative to control while (⇓) indicates down regulation of gene expression relative to control.

TABLE 2
CHANGES IN CYTOKINE AND GENE EXPRESSION

| Cytokine or | | | |
|---|---|---|---|
| Tissue Gene | | | |
| Expression | Maldextrin | K-NP-51 | L-NP-51 |
| Caudal (cdx-1) | ↓ | ↓ | ↑ |
| TNF-α | ↑ | ↓ | ↓ |
| iNOS | ↑ | ↓ | ↑ |
| Il-1 | ↓ | ↓ | ↓ |
| TGF-β | ↓ | ↓ | ↓ |

Together, these results suggest decreased host immune response against NP-51 in the intestinal tissues, which suggests that the lactic acid producing acteria strain NP51 is suitable for daily administration to the animals at the dosage used in this Example.

The description of the specific embodiments reveals general concepts that others can modify and/or adapt for various applications or uses that do not depart from the general concepts. Therefore, such adaptations and modifications should and are intended to be comprehended within the meaning and range of equivalents of the disclosed embodiments. It is to be understood that the phraseology or teiminology employed herein is for the purpose of description and not limitation. Certain terms with capital or small letters, in singular or in plural forms, may be used interchangeably in this disclosure.

### REFERENCES

All references mentioned in this application or listed below are incorporated by reference to the same extent as though fully replicated herein.
1. Chuang, L., K. G. Wu, C. Pai, P. S. Hsieh, J. J. Tsai, J. H. Yen, and M. Y. Lin. 2007. Heat-killed cells of lactobacilli skew the immune response toward T helper 1 polarization in mouse splenocytes and dendritic cell-treated T cells. Journal of Agricultural and Food Chemistry. 55:11080-11086.
2. Delcenserie, V., D. Martel, M. Lamoureux, J Amiot, Y. Boutin, and D. Roy. 2008. Immunomodulatory effects of probiotics in the intestinal tract Current Issues in Molecular Biology. 10:37-53.
3. Elam N A, Gleghorn JF , Rivera J D, Galyean M L, Defoor P J, Brashears M M, Younts-Dahl S M. 2003. Effects of live cultures of Lactobacillus acidophilus (strains NP45 and NP51 and Propionbacterium freudenreichii on performance, carcass, and intestinal characteristics, and Escherichia coli strain O157 shedding of finishing beef steers. J Anim Sci. 2003. 81: 2686-2698.
4. Stephens, T. P., G. H. Loneragan, E. Karunasena, and M. M. Brashears. 2007. Reduction of Escherichia coli O157 and Salmonella in Feces and on hides of feedlot cattle using various doses of a direct-fed microbial. Journal of Food Protection. 70:2386-2391.
5. Williams, P. 2007. Bacillus subtifis: A shocking message from a Probiotic. Cell Host & Microbe. 1:248-249.
6. Zanini, K., M. Marzotto, A. Castellazzi, A. Borsari, F. Dellaglio, and S. Torriani. 2007. The effects of fermented milks with simple and complex probiotic mixtures on the intestinal microblota and immune response of healthy adults and children. International Dairy Journal. 17:1332-1343.

What is claimed is:

1. A method of preventing or treating a gastrointestinal disease in a mammal, said method comprising the step of administering to said mammal an effective amount of at least one probiotic bacterium or derivative thereof, wherein said gastrointestinal disease is at least one member selected from the group consisting of Johne's Disease (JD) and Crohn's Disease (CD), and said at least one probiotic bacterium or derivative thereof is selected from the group consisting of live probiotic bacterium, inactivated probiotic bacterium and extract of probiotic bacterium.

2. The method of claim 1, wherein said mammal has contracted said gastrointestinal disease or has an increased susceptibility to infection by MAP as compared to the average susceptibility of mammals belonging to the same species.

3. The method of claim 1, wherein said at least one probiotic bacterium comprises at least one species belonging to the genus of Lactobacillus and at least one species belonging to the genus of Propionibacterium.

4. The method of claim 1, wherein said at least one probiotic bacterium comprises at least one strain selected from the group consisting of M35, LA45, NP51, L411, D3 and combination thereof.

5. The method of claim 1, wherein said at least one probiotic bacterium comprises at least one strain selected from the group consisting of P9, PF24, P42, P93, P99 and combination thereof.

6. The method of claim 1, wherein said at least one probiotic bacterium comprises the Lactobacillus strain NP51 and the Propionibacterium PF24.

7. The method of claim 2, wherein said effective amount is the amount of said lactic acid producing bacterium effective in reducing the MAP burden by at least 50% in at least one organ of said mammal, the organ being selected from the group consisting of liver, spleen and MLN.

8. The method of claim 2, wherein said effective amount is the amount of said lactic acid producing bacterium effective in reducing the MAP burden in the liver of said mammal by at least 80%.

9. The method of claim 2, wherein said effective amount is between 10⁵ and 10⁸ CFU of said lactic acid producing bacterium per mammal per day.

10. The method of claim 2, wherein said effective amount is about 10⁶ CFU of said lactic acid producing bacterium per mammal per day.

11. A method of treating a gastrointestinal disease at least partially caused by infection by Mycobacterium avium subsp. paratuberculosis (MAP), said method comprising the step of administering an amount of a lactic acid producing bacterium or derivative thereof to a mammal in need of said treatment, said amount being an effective amount in reducing the infectibility of said mammal by said Mycobacterium avium subsp. paratuberculosis (MAP), said at least one lactic acid producing bacterium or derivative thereof is selected from the group consisting of live lactic acid producing bacterium, inactivated lactic acid producing bacterium and extract of lactic acid producing bacterium.

12. The method of claim 11, said method further comprising a step of determining whether said mammal has contracted said gastrointestinal disease or whether said mammal has an increased susceptibility to infection by MAP.

13. The method of claim 11, wherein said gastrointestinal disease is at least one member selected from the group consisting of Johne's Disease (JD), Crohn's Disease (CD) and Inflammatory Bowel Disease (IBD).

14. The method of claim 11, wherein said lactic acid producing bacterium is at least one strain selected from the group consisting of M35, LA45, NP51, L411, D3 and combination thereof.

15. The method of claim 11, wherein said effective amount is an amount effective in increasing the frequency of cytotoxic T cells in the spleen of said animal by at least 5% 45 days after treatment as compared to the frequency of cytotoxic T cells in the spleen of untreated mammal of the same breed.

16. The method of claim 11, wherein said effective amount is an amount effective in increasing the levels of at least one pro-inflammatory cytokine in said mammal by at least 40% after a period of between 100 days to 180 days of treatment as compared to the levels of said at least one pro-inflammatory cytokine in untreated mammal of the same breed, said at least one pro-inflammatory cytokine being selected from the group consisting of IL-12, IFN-gamma and TNF-alpha.

17. The method of claim 11, wherein the effective amount is an amount effective in reducing the MAP burden in the liver of said animal by at least 80% after a period of between 100 days to 180 days of treatment as compared to the MAP burden in the liver of untreated mammal of the same breed.

18. The method of claim 11, wherein said effective amount is between 10⁵ and 10⁸ CFU of said lactic acid producing bacterium per mammal per day.

19. The method of claim 11, wherein said effective amount is about 10⁶ CFU of said lactic acid producing bacterium per mammal per day.

20. A method for preventing infection caused by a pathogenic microorganism, said method comprising the step of administering, not later than 4 weeks after birth, an amount of a lactic acid producing bacterium to a mammal, said amount being an amount effective in reducing the infectibility of said mammal by Mycobacterium avium subsp. paratuberculosis (MAP).

21. The method of claim 20, wherein said mammal is a ruminant.

22. The method of claim 20, wherein said gastrointestinal disease is at least one member selected from the group consisting of Johne's Disease (JD), Crohn's Disease (CD) and Inflammatory Bowel Disease (IBD).

23. The method of claim 20, wherein said lactic acid producing bacterium is at least one strain selected from the group consisting of M35, LA45, NP51, L411, D3 and combination thereof.

24. The method of claim 20, wherein said effective amount is between 10⁵ and 10⁸ CFU of said lactic acid producing bacterium per mammal per day.

25. The method of claim 20, wherein said effective amount is about 10⁶ CFU of said lactic acid producing bacterium per mammal per day.

26. The method of claim 25, wherein said effective amount is increased to between 5×10⁸ and 5×10⁹ CFU of said lactic acid producing bacterium per mammal per day starting from about 40 days prior to the time when said animal is to be slaughtered.
